(19) United States
(12) Patent Application Publication
(10) Pub. No.: US 2026/0098250 A1
(43) Pub. Date: Apr. 9, 2026

(54) METHOD AND DEVICE AT LEAST FOR THE CONTROLLED MOVEMENT OF GAMETES AND/OR ZYGOTES AND/OR EMBRYOS IN FLUIDS

(71) Applicant: ASOCIACIÓN CENTRO DE INVESTIGACIÓN COOPERATIVA EN NANOCIENCIAS "CIC NANOGUNE", Donostia - San Sebastian (ES)

(72) Inventor: Mariana MEDINA-SÁNCHEZ, Dresden (DE)

(73) Assignee: ASOCIACIÓN CENTRO DE INVESTIGACIÓN COOPERATIVA EN NANOCIENCIAS "CIC NANOGUNE", Donostia - San Sebastian (ES)

(21) Appl. No.: 19/109,850

(22) PCT Filed: Sep. 6, 2023
(86) PCT No.: PCT/EP2023/074481

§ 371 Date: Mar. 7, 2025

(30) Foreign Application Priority Data
Sep. 9, 2022 (DE) 10 2022 122 900.9

(51) Int. Cl. C12N 13/00

(52) U.S. Cl. CPC C12N 13/00

(57) ABSTRACT

A method, as can be applied, for example, in in vivo reproduction and in healthcare, and to a device for realizing the controlled movement. The device can be used in a considerably less invasive manner and significantly higher implantation rates can be realized using the method. By the method, gametes and/or zygotes and/or embryos are releasably connected to one or more carriers, and the carriers, with or without gametes and/or zygotes and/or embryos, are moved in a directed manner autonomously or by external influences. The device includes at least one carrier having maximum dimensions in all spatial directions of 500 μm, which carrier is composed at least primarily of biocompatible materials.

The invention concerns the fields of materials science, biology, and medicine and relates to a method at least for the controlled movement of gametes and/or zygotes and/or embryos in fluids, as can be applied, for example, in in vivo reproduction and in healthcare, wherein in addition to the controlled movement of gametes and/or zygotes and/or embryos, a safe development of the gametes and/or zygotes and/or embryos can, for example, also be realized, and to a device for realizing the controlled movement.

Infertility is a problem that affects 48.5 million couples, approximately 11% of populations, at a reproductive age worldwide. The possible causes in women are ovulation dysfunctions, tubal obstruction, endometriosis, and uterine and/or cervical factors.

In men, infertility is normally caused by a poor sperm quality, which presents in a low motility or abnormal morphology or in a low quantity of sperm. This reduces the opportunities for fertilizing the egg cell in vivo.

The known treatment methods for infertility include a cost-effective and minimally invasive hormonal stimulation or an intrauterine insemination, in which spermatozoa are injected into the uterus during ovulation.

Furthermore, methods of in vitro fertilization (IVF) or intracytoplasmic sperm injection (ICSI) are known for improving the chances of pregnancies. These methods are especially indicated where a tubal obstruction infertility, or a serious male infertility is diagnosed.

Due to the improved protocols and better gamete selection techniques, the use of these methods has rapidly increased and now achieves fertilization rates of approximately 95%.

However, the subsequent embryo transfer is still at the critical stage, with only 32% of the cases resulting in clinical pregnancies. In addition, the implantation rates per embryo remain very low (˜17%), and the procedure must often be repeated several times, without success, which entails high economic and social costs.

Even with the first three embryo transfers of a genetically tested euploid embryo in the blastocyst stage and the treatment of women having an anatomically normal uterus, the implantation rates are only between 60.3%-69.9%.

Possible causes for these low implantation rates, and therefore pregnancy rates, of transferred embryos that were obtained by IVF and ICSI can be the stress to which the gametes are exposed during the in vitro preparation and cultivation steps or lifestyle factors of the couple undergoing treatment, illnesses, uterine or endometrial anomalies, embryonic factors, or even different techniques in the IVF lab or during the embryo transfer.

Despite this, no apparent explanation is found in most cases. For these medical problems, other diagnostics, such as hysteroscopy and endometrial biopsy, or the treatment of endometrial injuries, changes to the simulation protocol, blastocyst transfer, assisted hatching, and pre-implantation screening, could help.

One promising method for improving the implantation rates in RIF (repeated implantation failure) appears to be the intrafallopian transfer of gametes or zygotes or of the egg cell inseminated in vitro (embryo) back into the fallopian tube by laparoscopy, also referred to as gamete/zygote intrafallopian transfer (GIFT/ZIFT). It is assumed that the advantage of this method is that the suitable physiological environment for the embryonic development and an optimal synchronization between embryonic and endometrial preparation are present. However, the reproducibility of these methods is very low and depends on the abilities of the operating surgeon and on the cultivation conditions before the gametes or the embryo are transferred back into the fallopian tube or the uterus.

In addition, this method is very invasive and requires a surgical procedure and anesthesia in order to insert the macroscopic imaging and manipulation tools for the laparoscopy into the female body. This can result in side effects such as fallopian tube trauma, inflammation, tubal pregnancies, infections, or intraluminal pathologies.

Nevertheless, this treatment appears to have resulted in comparatively high pregnancy rates of 39.8% in patients having high-order RIF (prolonged infertility duration and a high number of failed standard IVF-ET cycles), so that this method could possibly have yet unutilized potential.

Other methods are known for improving the described problems, in particular for improving the mobility of motile cells, such as spermatozoa. According to DE 10 2012 212 427 A1, a method is known in which one or more motile cells are introduced into or attached to one magnetic particle or multiple magnetic particles and, subsequently, the magnetic particles with the motile cells introduced thereinto or attached thereto are moved in a directed manner by an application of an external magnetic field.

With this method, the activity and guided mobility of motile cells is improved and the movement of the motile cells is guided in a desired direction.

Methods of this type are not suitable for immobile cells.

Immobile cells are, for example, spermatozoa which have lost their mobility or egg cells in general.

The movement of an egg cell occurs in that the funnel-shaped fimbria sweeps over the ovary and picks up the egg cell during ovulation. The fallopian tube thereby contracts rhythmic movements. The transport of the egg cell in the fallopian tube is handled by tiny cilia (ciliary cells). They push the egg cell in the direction of the uterus (Wikipedia, Stichwort Bewegung einer Eizelle [Keyword Movement of an Egg Cell]).

A method is known in which an egg or an embryo is provided with a layer of magnetic particles and, by application of a magnetic field using a permanent magnet or an electromagnet, the egg or the embryo is stabilized in the uterus (U.S. Pat. No. 6,695,766 B4). The transport of the egg or of the embryo with the layer of magnetic particles into the uterus takes place by means of a catheter. The magnetic particles can have a diameter of 0.1 μm to 200 μm. These magnetic particles are brought together with the egg or the embryo so that the reactive groups at the surface of the magnetic particles can react with reactive groups on the surface of the egg or of the embryo and thus achieve the adhesion of the magnetic particles to the surface of the egg or of the embryo.

Also known is a method for producing mobility in immobile cells in which the immobile cell, immobile spermatozoa, is connected to a microstructure, wherein the microstructure is composed at least partially of magnetic materials and a non-reciprocal movement of the microstructure with the immobile cell is executed by means of a time-varying, three-dimensional external magnetic field (DE 10 2014 201 760 A1).

Furthermore, from U.S. Pat. No. 6,050,935 A, a container arrangement is known for intravaginal fertilization and embryo transfer, which container arrangement is used during intravaginal fertilization. For this purpose, the container is inserted into the vaginal vault.

The container comprises a container body with an opening for the insertion of a growth medium, one or more egg cells and spermatozoa, and for the subsequent extraction of an embryo or multiple embryos using an extraction catheter. The container body comprises a main chamber for accommodating the growth medium, the egg cells and the sperm, and a microchamber for collection for the extraction of one or more embryos. The container is encased by a capsule of soft, elastic material. A loop on a portion of the capsule adjusts the length to the anatomical conditions. The microchamber comprises a channel which accommodates the catheter tip in order to facilitate the extraction of the embryos and, at the same time, to eliminate the risk of injury to the embryos. The microchamber enables the microscopic inspection of the embryos in situ before the transfer into the uterine cavity.

A disadvantage of the known solutions from the prior art is that, on the one hand, invasive methods are known and applied for the implantation of embryos and, on the other hand, relatively low implantation rates, and therefore pregnancy rates, of transferred embryos are still achieved. Also, comparatively large devices are used in the known methods, which devices can lead to injuries to the embryos and/or to tissue, or even to the implantation of the embryo at an improper site, which results in an ectopic pregnancy. Likewise, the low precision of the known methods and devices leads to the fertilization and birth of multiple embryos, which is also not desirable since multiple pregnancies constitute a high risk for mother and baby.

The object of the present invention is to specify a method and a device at least for the controlled movement of gametes and/or zygotes and/or embryos in fluids, in which the device can be used in a considerably less invasive manner and significantly higher implantation rates, and therefore pregnancy rates, can be realized using the method.

The object is attained by the invention disclosed in the patent claims. Advantageous embodiments are the subject of the dependent claims, wherein the invention also includes combinations of the individual dependent patent claims within the meaning of an and-operation, provided that they are not mutually exclusive.

In the method according to the invention at least for the controlled movement of gametes and/or zygotes and/or embryos in fluids, gametes and/or zygotes and/or embryos are releasably connected to one or more carriers, and the carriers, with or without gametes and/or zygotes and/or embryos, are moved in a directed manner autonomously or by means of external influences.

Advantageously, a connection between gametes and/or zygotes and/or embryos and the carrier or carriers is realized, which connection can be realized and/or released by the use of physical or chemical means.

Likewise advantageously, carriers or components of the carriers, with or without gametes and/or zygotes and/or embryos, are used which are mechanically or digitally connected using a force-and/or form-fitting, releasable or non-releasable contact to one or more devices arranged ex vivo, wherein it is more advantageous if devices arranged ex vivo for the guidance and/or movement and/or loading of the carriers are used before the in vivo transfer.

It is also advantageous if carriers are used which move autonomously, with multiple carriers moving independently of one another in the same or different directions or moving as a swarm in the same direction.

And it is also advantageous if the carriers are moved by means of an external physical field, such as a magnetic or ultrasound field, or by means of chemical, thermal, or physical stimuli.

It is also advantageous if the position and movement of the carriers are monitored by means of imaging methods.

It is furthermore advantageous if parameters from the environment of the carriers are detected, transmitted, and monitored in situ by means of sensors or actuators located on or in the carriers, and/or changes in the parameters are realized based on detected parameters from the environment of the carrier.

The device according to the invention at least for the controlled movement of gametes and/or zygotes and/or embryos in fluids contains at least one carrier having maximum dimensions in all spatial directions of 500 μm, which carrier is composed at least primarily of biocompatible materials.

Advantageously, the at least one carrier has dimensions in all spatial directions of 10 nm to 100 82 m, advantageously of 1000 nm to 80 μm, more advantageously of 10 nm to 1000 nm.

Likewise advantageously, the biocompatible materials of the carriers are fully degradable in the body.

It is furthermore advantageous if the outer shell of the carriers is entirely composed of biocompatible materials.

And it is also advantageous if, as biocompatible materials, hydrogels, such as gelatin, methacrylates, collagen, silk, alginates, or biodegradable metal oxides, or metallic alloys such as FeMgSi or FePt, with or without magnetically hard materials such as NdFeB, CrO₂, or BaFe₁₂O₁₉, or superparamagnetic iron oxide nanoparticles, such as SPIONs or iron-platinum nanoparticles are present, or other materials are coated with biocompatible materials, such as surface coatings made of proteins, gold, or polymers such as parylene or poly(ethylene glycol) diacrylate (PEGDA).

It is also advantageous if the carriers comprise components that are holders, grippers, slings, catheters, cavities for the releasable connection to gametes and/or zygotes and/or embryos, sensors and/or actuators.

It is likewise advantageous if the carriers comprise propulsion devices for autonomous movement.

It is furthermore advantageous if the carriers are microrobots or nanorobots.

And it is also advantageous if the carriers comprise materials with which the carriers are moved by external or local influences.

It is also advantageous if the carriers contain organic, inorganic, and/or magnetic materials.

And it is also advantageous if, on or in the carriers, sensors or actuators are present which detect, transmit, and monitor in situ parameters from the environment of the carriers and/or realize changes in the parameters based on detected parameters from the environment of the carrier.

It is furthermore advantageous if the carrier or carriers is/are connected to devices arranged ex vivo for the guidance and/or movement and/or loading of the carriers before the in vivo transfer, wherein the connection is realized via mechanical devices or digitally.

It is likewise advantageous if the carriers comprise functional materials and/or biomolecules for the protection and/or the growth of the gametes and/or zygotes and/or embryos, wherein it is more advantageous if the carriers comprise antioxidants, maturation factors, nutrients, protective molecules for reducing thermal and mechanical stresses.

With the present invention, it is for the first time possible to specify a method and a device at least for the controlled movement of gametes and/or zygotes and/or embryos in fluids, in which the device can be used in a considerably less invasive manner and significantly higher implantation rates, and therefore pregnancy rates, can be realized using the method.

This is achieved through a method for the controlled movement of gametes and/or zygotes and/or embryos in fluids, in which gametes and/or zygotes and/or embryos are releasably connected to one or more carriers.

Gametes, also referred to as sex cells or germ cells, are specialized cells, two of which join to form a zygote in sexual reproduction.

A zygote is a eukaryotic diploid cell that forms by the fusion of two haploid sex cells in sexual reproduction, usually from an egg cell and a sperm.
An embryo is a living organism in the early phase of its development.

The gametes and/or zygotes and/or embryos which are to be moved according to the invention can be from humans or animals.

The connection and also the release of the connection between the gametes and/or zygotes and/or embryos and the one or more carriers can take place by the use of physical and/or chemical means.

Physical means can be mechanical means such as holders, grippers, slings, catheters, cavities on or in the carrier.

Likewise, nano- to micro-actuators can be present on or in the carrier material, for example with gripper arms or gripper fingers, which can be opened or closed by electrical, mechanical, thermal, and/or chemical stimuli. Nano- to micro-actuators of this type have advantageous dimensions on the scale of the gametes and/or zygotes and/or embryos.
It can thereby be advantageous that the carrier also comprises cavities or capsules, if possible with permeable or semi-permeable walls, into which the gametes and/or zygotes and/or embryos or other materials can be received.

Carriers that are attached to microcatheters in a force fit and/or form fit can be particularly advantageous as mechanical means. The microcatheter is thereby connected to devices for the guidance and/or movement and/or loading of the carriers before the in vivo transfer. A carrier according to the invention can therefore be inserted into the body by a conventional cannula or an egg cell transfer catheter. As a result of the at least micro-dimensions of the catheter and carriers, it is also possible to reach complex and sensitive regions of the body, such as the fallopian tube.

This connection of the microcatheter to the outside of the body can advantageously be realized via mechanical devices or digitally.
In such a case, the carrier according to the invention can be releasably or non-releasably attached to the mechanical means, such as the microcatheter.

Additionally, freely movable carriers that can move autonomously can be present as unattached carriers.

Carriers of this type can be mobile grippers and/or capsules, or can be present in the form of rods and/or cylinders.

The carriers can also be subdivided, for example into different modules inside of the same carrier, whereby different components (gametes/zygote/embryo, magnetic particles, contrast media, antioxidants, etc.) can be held and even be held separately.

The transfer of these carriers into the body can take place via a conventional cannula or an embryo transfer catheter into the uterus.

These mobile, unattached carriers can, for example, be moved in a directed manner by the influence of an external field. Thus, the carriers, with or without gametes and/or zygotes and/or embryos or other materials, can move for example by swimming, crawling, ambulating, or rolling without a mechanical connection to the outside of the body, into the desired regions and, in particular, also the difficult-to-reach and/or sensitive regions, such as the fallopian tube, for example.

Advantageously, the movement of the carriers is monitored in real time by means of bioimaging with a highest possible spatial and temporal resolution and is guided accordingly.

Chemically, the connection can be realized via functional groups and/or gels, for example by incorporating the carrier into hydrogels, coupling to stimuli-sensitive linkers, coupling via hydrophilic/hydrophobic interactions, via hydrodynamic or electrostatic or magnetic or acoustic forces.

The release of the connection between the carrier and at least the gametes and/or zygotes and/or embryos can be achieved by the loosening of the physical connections or dissolution of the chemical bonds, but also in that carrier materials are used, the degradation, and specifically the biodegradation, of which are realized in the body. The degradation rate should thereby be adapted to the respective transfer of the gametes and/or zygotes and/or embryos, including the destination and the dwell time necessary therefor, and should be guided, for example, by the composition of the local environment or locally present proteases, or by external influences, such as the use of local temperature increase. Once the gametes and/or zygotes and/or embryos have reached the destination, the biodegradable carrier material can then dissolve.

According to the invention, the carriers, with or without gametes and/or zygotes and/or embryos, are then moved in a directed manner autonomously or by means of external influences.

The carriers can thereby initially also be moved or move outside of the body without gametes and/or zygotes and/or embryos, and can be introduced into the body using other devices, such as cannulas or catheters, in order to then take up and secure the gametes and/or zygotes and/or embryos and transport them to the desired location.

Likewise, the carriers can be moved by local influences such as chemotaxis, thermotaxis, thigmotaxis, or rheotaxis, and/or the movement thereof can be guided in a directed manner. The carriers can also be guided by external physical fields such as magnetic field, ultrasound and/or infrared light.

However, the carriers can also already be connected to the gametes and/or zygotes and/or embryos outside of the body and then be collectively transported into the body. This can also take place via other means, such as cannulas or catheters, or the carriers, with the gametes and/or zygotes and/or embryos, move autonomously or are moved by means of external influences.

For example, the gametes and/or zygotes and/or embryos can be provided with a carrier in vitro and then be transported through the cervix to the entrance of the uterus, for example, using a nonsurgical embryo transfer set (NSET) or a flexible cannula.
At the desired location, for example in the uterus, the carriers can then be separated from the gametes and/or zygotes and/or embryos by external or local chemical or physical triggers. External influences of this type can advantageously be external hydrodynamic, magnetic, electrostatic, and/or acoustic fields.

Likewise advantageously, the position and movement of the carriers can be monitored by means of imaging methods or external sensors. The carrier can thereby trigger local reactions or biomarkers which can subsequently be detected in biological fluids. This detection can be carried out using external rapid test sensors or PCR, for example. Similarly, a local change in shape or activation of a “report” part of the carrier may occur, which can be detected by magnetic resonance methods or PET or other imaging methods, for example.

Furthermore, the device according to the invention for the controlled movement of gametes and/or zygotes and/or embryos in fluids contains at least one carrier having maximum dimensions in all spatial directions of 500 μm, which carrier is composed at least primarily of biocompatible materials.

Advantageously, the maximum dimensions of the carriers in all spatial directions are from 10 nm to 100 μm, advantageously from 1000 nm to 80 μm, more advantageously from 10 nm to 1000 nm.

Furthermore, the size and materials of the carrier are, on the one hand, determined by the gametes and/or zygotes and/or embryos that are to be transported; yet the carriers are also composed at least primarily of biocompatible materials which can themselves sense the microenvironment of the gamete and/or zygote and/or embryo and react to said microenvironment, for example by changing the shape or the surface properties, for example in order to release necessary substances that ensure a secure transport and a secure development of the gametes and/or zygotes and/or embryos during the transfer into the fallopian tube or into the uterus. Likewise, the carrier materials are advantageously permeable to nutrients, antioxidants, and growth factors that are able to and intended to enter the body in vivo through the carrier materials or enter the carrier from the fallopian tube fluid.

The maximum dimensions of the carriers do not exceed in any spatial directions the minimum dimensions of the fallopian tubes, with an approximate 500 μm diameter.

In any case, the carrier has the smallest possible dimensions; however, carriers with dimensions up to approximately 500 μm can also be possible, since the regions in which the carrier must move are adaptable tissues which are folded and therefore also stretchable.
Advantageously, the carriers have a similar size as the gametes and/or zygotes and/or embryos, in order to enable access to the narrowest regions of the fallopian tube without damaging the surrounding tissue.
The carriers have dimensions and shapes such that they protect the gametes and/or zygotes and/or embryos during the transport, for example to the ampoule, so that they can develop under the most natural possible conditions until they have reached the uterine endometrium, the uterine mucous lining in the interior of the uterus. This can be achieved, for example, with capsules in which the gametes and/or zygotes and/or embryos, but also other materials, are enclosed during the transport.

It is also particularly advantageous if the outer shell of the carriers is entirely composed of biocompatible materials. This is particularly important if a carrier is used that is intended to remain in the body and/or to be degraded there. If the carrier is able to and intended to be removed from the body, other medically harmless materials can also be used.

Likewise advantageously, the biocompatible materials of the carrier are fully degradable in the body.

According to the invention, the degradation of the carrier materials takes place when the carrier has performed its function and is not removed from the body by other measures, methods, or devices.

Biocompatible materials of this type are in particular hydrogels, such as gelatin, methacrylates, collagen, silk, alginates, or biodegradable metal oxides, which are all as biodegradable, permeable, and/or soft as possible and carry out a gentle interaction with the surrounding tissue in the body.

Other biocompatible materials are, for example, metallic alloys such as FeMgSi or FePt, with or without magnetically hard materials such as NdFeB, CrO₂, or BaFe₁₂O₁₉, or superparamagnetic iron oxide nanoparticles, such as SPIONs or iron-platinum nanoparticles. Or, other materials can be used which are coated with biocompatible materials, such as surface coatings made of proteins, gold, or polymers such as parylene or poly(ethylene glycol) diacrylate (PEGDA).

Furthermore, the carriers can, as needed, comprise materials which are or contain antioxidants, hormones, medications and/or which are permeable for the exchange of nutrients from the secretory cells in the body, and specifically in the fallopian tube. Materials of this type can also be factors for the epigenetic regulation, the protection of the immune system, the protection against oxidative stress and heat stress, or adjuvant substances for the splitting and development of the embryo (embryotropic factors, growth factors).

As a result of the materials used for the carriers, the friction of the carrier with the gametes and/or zygotes and/or embryos is significantly reduced, whereby potential damage and cytotoxicity and stress for the gametes and/or zygotes and/or embryos are avoided. Also, the passage/exchange of the nutrients and of the secreted fallopian tube fluid for embryonic development and transport is, advantageously simultaneously, enabled or supported.

Advantageously, the carrier or carriers comprise(s) functional materials and/or biomolecules for the protection and/or for the growth of the gametes and/or zygotes and/or embryos. Functional materials of this type can also be nanosensors for the monitoring of environmental parameters of the microenvironment, such as oxygen content, blood glucose level, growth factors etc., which can also be regulated in situ by the nanoactuators of the carrier or materials contained therein.

The carriers made of the biocompatible, and advantageously biodegradable, materials can be produced using methods of two-photon lithography or by means of methods for producing strained materials (strain engineering) or by means of 3D or 4D printing technologies.

The carriers can also be produced by means of droplet microfluidics. This production method offers the advantage that gametes and/or zygotes and/or embryos can be directly introduced at the same time into individual droplets of biodegradable or stimuli-sensitive polymers.
Other possibilities of producing the carriers exist with chemical synthesis or with template-based methods.

These droplets can later be transferred into the body, where they are moved in a directed manner by means of ultrasound waves or by magnetic propellers or by local stimuli such as temperature gradients, chemical gradients, flux.

The magnetic propellers can be placed in microfluidic channels and, through the generation of differential currents, thus facilitate the capture of gametes and/or zygotes and/or embryos in the droplets. However, the capture can also be realized and/or facilitated by self-assembly techniques or by the utilization of capillary forces, hydrodynamic forces, or hydrophilic/hydrophobic interactions. The capture of the gametes and/or zygotes and/or embryos can also be realized through the coupling or loading of the carriers in vitro using optical, magnetic, or acoustic tweezers.

Advantageously, the carriers can comprise propulsion devices for autonomous movement. In this case, these can advantageously be microrobots or nanorobots.

However, the use of magnetic propellers as part of the carriers can also result in an autonomous movement of the carriers with or without gametes and/or zygotes and/or embryos.

As carriers in the form of microrobots, structural materials such as PEGDA can be used, or hollowed hydrogel tubes or 3D-printed elastomers such as silicone, polyurethane, polyolefin, styrenic block copolymers.

As materials for the propulsion of the microrobots, Fe, FePt can be used for the magnetic movement, for example, or electroactive polymers, such as polypyrrole or other hydrogels that react in a stimulating manner.
Some inorganic layers made of or comprising alloys or core-shell nanoparticles, for example of Au or Pt, can also be used for the sensors; or, for the imaging, Au-based nanomaterials or nanomaterials as quantum dots (QDs) can be used, or Ag can be used to prevent the formation of biofilms.

Due to the autonomous movement, the carriers themselves can realize a slight current, as a result of which the carriers are capable of moving in the fluids in the body, which are usually viscoelastic media. And, in particular, they can also move against the current or against back flows, such as in the fallopian tube due to the peristaltic movement thereof and ciliary beating, for example.

If the carriers comprise magnetic materials, they can also be transported via external magnetic fields. This can also take place if the carriers are in droplet form.

Likewise, the carriers can contain fluids with which a directed movement of the carriers can be realized by means of an influence of ultrasound from outside of the body or by an artificial and/or biohybrid flagellar-like propulsion.

Furthermore, carriers in droplet form can also be multifunctional, in that additional means can be integrated during the formation thereof. These means can be active substances that are present in microcapsules, as well as magnetic materials in pearls and/or infrared reporters, reflectors or absorbers, in order to be able to track the carriers during the movement thereof, and in particular in vivo.

The carriers can also contain antioxidants and estrogen, in order to protect the gametes and/or zygotes and/or embryos during the transport thereof.

In addition, the use of metal-organic materials or oxygen-sensitive particles as components of the carriers is possible, in order to monitor the metabolism of gametes and/or zygotes and/or embryos during the journey thereof in vivo. Such materials and components of the carrier can also be referred to as nanosensors or nanoactuators.

Nanosensors or nanoactuators of this type can measure the concentrations of, in particular, harmful metabolites, the blood glucose level, and the oxidation condition of the embryo, and can release in situ medications or substances such as antioxidants, nutrients, and growth factors in order to fight harmful metabolites.
Likewise, via the nanosensors or nanoactuators, parameters from the microenvironment, such as the pH value, secreted proteins, growth factors, reactive oxidative species (ROS), hormones, substances secreted from the fallopian tube, for example, can be monitored in situ and a corresponding reaction of the carrier can be activated, such as, for example, the local release of medications/molecules/antioxidants/growth factors or the change in shape thereof, for example for the release of the gametes and/or zygotes and/or embryos. In addition, detected parameters of this type can be identified by bioimaging or by biofluid analysis outside of the body, such as via lateral flow sensors or PCR, for example.

Also advantageously, the carriers can comprise components that are holders, grippers, slings, catheters, cavities for the releasable connection to gametes and/or zygotes and/or embryos, and/or sensors and/or actuators.

These components can thereby be directly actuated by the carrier via on-board power supplies, but also externally via fields.

This could also result in the carriers in the future being capable of sensing their environment, analyzing the environment and the gametes and/or zygotes and/or embryos during the journey in the body, and obtaining deeper insights into the function and structure of the female reproductive tract in the different stages of the estrous cycle.

Similarly, with the carriers according to the invention, sensors can be brought into the region of the gametes and/or zygotes and/or embryos, which sensors can serve to conduct the in situ monitoring of embryonic development; or mechanical, optical, and/or electrical actuators are present for cell stimuli or for in situ molecule release. Sensors of this type can not only monitor the development of the embryo, but also measure parameters of the environment such as the local pH value, the temperature, the blood glucose level, etc., both in vivo and, in particular, also in the fallopian tube, and transmit the measured values outside of the body.

Further advantageously, the carrier or carriers can be mechanically or digitally connected to devices arranged ex vivo for guiding the movement and/or the components on the carriers.

The carriers according to the invention can be attached outside of the body, for example to catheters or tethers, and can move or be moved in the body in said attachment or unattached. The movement can be in a swimming, rolling, gliding, and/or ambulating manner, or can be a peristaltic movement or movement triggered by a traveling wave of the surrounding fluid.

The external influences are advantageously external magnetic fields or external ultrasound or a combination thereof, wherein said external influences can, at the same time, be utilized for the application of imaging methods.

With the solution according to the invention, gametes and/or zygotes and/or embryos of the highest quality can be non-invasively transported and released in vivo. The carriers used for this purpose are advantageously multifunctional microrobots with maximum dimensions in all spatial directions of 500 μm that are at least primarily composed of intelligent, biocompatible materials and have been produced by means of advanced microtechnologies.

As a result of the solution according to the invention, the fertilization and embryo implantation in vivo can be supported and improved.

Similarly, the solution according to the invention can be successfully applied in particular in difficult-to-access regions of the reproductive system, such as the fallopian tubes. The fallopian tube is an organ with an important role in the migration/support of the gametes and zygotes during fertilization. Likewise, the fallopian tube is important during the development and the transport of an embryo to the uterus, and specifically also precisely when the endometrium is ready for the embryo implantation.
The known methods and devices have previously not targeted the fallopian tube, which is a tiny and intrinsic organ that is difficult to reach.

In contrast to the established in vitro techniques, assisted reproductive methods can be applied in vivo with the proposed invention. The method and the device can also be expanded to include other medical scenarios, for example for the delivery of medications to treat illnesses such as cancer, endometriosis, and/or other problems in female and male reproductive medicine, and/or for the application to other organs, for example for the extraction of samples for diagnosis or for local diagnosis.

The carriers according to the invention, which are advantageously nano-to microrobots,
i) have the ability to reliably capture and secure the gamete and/or zygote and/or embryo during the transport between different environments;
ii) enable access of the secreted molecules, either by the ciliary cells of the fallopian tube or by the embryo itself;
iii) are biocompatible and/or advantageously biodegradable;
iv) are capable of moving in the fluids, which are mostly viscoelastic media, and against back flows in the fallopian tube; and
v) do not damage the fallopian tube, which is a very sensitive organ;
vi) enable the in situ detection of the reproductive organ and of the local microenvironment of the gametes and/or zygotes and/or embryos;
vii) can adapt their function (for example, the release of molecules, change in shape, permeability rate, etc.) according to the sensed microenvironment and the health of the gametes and/or zygotes and/or embryos;
viii) can potentially carry medications, for example to correct genetic defects of the gametes and/or zygotes and/or embryos during the transport or to halt the development thereof at an early stage if a noticeable, incurable dysfunction of the embryo is present.

As components on the carrier, microcatheter tools can, for example, be present which release the gametes and/or zygotes and/or embryos at the desired positions by externally or internally triggered mechanisms, but which can also be used to negotiate the complex turnings of the fallopian tube in a minimally invasive manner.

The carriers according to the invention can be introduced into the uterus or the fallopian tube through the vagina by means of a cannula or a catheter. There, they can then capture and transport gametes and/or zygotes and/or embryos for example by means of spherical microgrippers or capsule-like embryo carriers.

A major advantage of the solution according to the invention is that specifically gametes and/or zygotes, that is, sex cells and young embryos, in addition to older embryos, can be moved in a directed manner and can also be used for the gamete intrafallopian transfer (GIFT) or the zygote intrafallopian transfer (ZIFT). In addition to the known stabilization of embryos in the uterus, it is now also possible to move gametes and/or zygotes and/or embryos in a directed manner with little to no invasiveness. According to the invention, this directed movement can take place into the fallopian tube.

The carriers used for this purpose according to the invention, advantageously microrobots, do not need to remain in the body and, during contact with the gametes and/or zygotes and/or embryos, also do not need to be in direct contact. The gametes and/or zygotes and/or embryos can, for example, be present in a capsule which is then in direct contact with the carrier.

Additional advantages of the solution according to the invention over laparoscopy and intrauterine embryo transfer are:
Less invasive to non-invasive
The carriers according to the invention have smaller dimensions than current surgical tools.
They can be tethered, for example as robotic catheters, or as untethered, like swimming, rolling, gliding, and/or ambulating nano-to microrobots.
Selection of materials
The carriers according to the invention are biocompatible and advantageously biodegradable, whose degradation time, for example, can be adapted to the time required for the transfer of gametes and/or zygotes and/or embryos to the desired location.
Further functional possibilities
The carriers according to the invention can also be loaded with other functional materials and/or biomolecules in order to support embryonic development (for example, antioxidants, maturation factors, nutrients, protective molecules for the reduction of thermal and mechanical stresses).
Additional components
The carriers according to the invention can also be equipped with mechanical, optical, and/or electrical micro- or nanosensors or micro- or nanoactuators.

The advantages of the present invention have a highly relevant effect above all in the case of multiple failed embryo implantations where conventional in vitro fertilization methods are used and the embryo transfer occurs at the uterine site without a resulting pregnancy.

Due to the gentle interaction of the carriers according to the invention with the surrounding tissue in the body, compared with the real and unwieldy manipulation and imaging tools that are used nowadays in different steps of the assisted reproduction process, starting with the egg cell extraction and the further uterus implantation of the embryo or the laparoscopy-based intrafallopian transfer, can significantly improve implantation rates, and therefore the pregnancy outcomes.

Furthermore, it can be ensured according to the invention that only a high-quality embryo is connected to the carrier and introduced into the fallopian tube.

To ensure that the embryos/gametes are of high quality, and also retain said quality during the transport, the carrier can contain nanosensors which can measure, and in some cases also regulate, the different variables of the embryonic development process.

The present invention additionally has the advantage that essentially no non-biocompatible materials are used, or that said materials do not come into direct contact with the gametes and/or zygotes and/or embryos. Since the carriers can in any case be released from the gametes and/or zygotes and/or embryos according to the invention, even non-biocompatible materials of the carriers thus do not have an adverse influence on the gametes and/or zygotes and/or embryos, and can be removed from the body without a problem.

With the solution according to the invention, higher implantation rates can be realized, and the pregnancy outcomes can thereby be considerably improved.

The invention is explained below in greater detail with the aid of several exemplary embodiments.

### EXAMPLE 1

As a carrier, a microcatheter having dimensions of 100 μm diameter is produced by means of 3D printing from PEGDA as biocompatible material which forms the outer shell of the microcatheter. A cavity for an embryo and a micropump and a sensor for the optical/electronic monitoring of the embryo are integrated into the microcatheter.

Using the micropump, an embryo is gently drawn into the cavity of the microcatheter with the ambient fluid and nutrients outside of the body and is then introduced into the cervix through the vagina using a cannula. The microcatheter is subsequently moved in a directed manner to the uterine mucous membrane by ultrasound. With the aid of the sensor and external imaging devices, the position and the condition of the embryo are monitored until the arrival at the desired location.

After the positioning of the embryo, which is carried out without anesthetics or with local anesthetics and for which approximately 1 h is needed, the catheter can be removed.

### EXAMPLE 2

As a carrier, a microcapsule having a diameter of 500 μm is produced from gelatin as biocompatible material by means of droplet fluidics.

Both spermatozoa and also an egg cell, together with nanopropellers and nanosensors made of a magnetic material and also nutrients, are introduced into the microcapsule by means of droplet fluidics.

The microcapsule is then moved via external magnetic fields into the uterus through the vagina, and ultimately into the fallopian tube. At this site and at this point in time, the degradation of the biocompatible material begins, so that the spermatozoa and the egg cell are released, and the fertilization of the egg cell takes place during the luteal phase of the estrous cycle.

The resulting zygote can then develop into an embryo under more natural conditions and can reach the uterus in perfect synchronization with the endometrium preparation, in that the zygote migrates further to the uterus due to the peristalsis of the fallopian tube and can implant itself into the mucous membrane.

The zygote is thus positioned naturally, without anesthetics, but this can also be carried out under local anesthesia.

In approximately 1 hour, and together with the magnetic material, the biocompatible material of the microcapsule is removed from the body with the surrounding fluid of the fallopian tube.

1. A method at least for the controlled movement of gametes and/or zygotes and/or embryos in fluids, comprising:
gametes and/or zygotes and/or embryos are releasably connected to one or more carriers, and the carriers, with or without gametes and/or zygotes and/or embryos, are moved in a directed manner autonomously or by external influences.

2. The method according to claim 1, in which a connection between gametes and/or zygotes and/or embryos and the carrier or carriers is realized, which connection can be realized and/or released physically or chemically.

3. The method according to claim 1, in which carriers or components of the carriers, with or without gametes and/or zygotes and/or embryos, are used which are mechanically or digitally connected using a force- and/or form-fitting, releasable or non-releasable contact to one or more devices arranged ex vivo.

4. The method according to claim 3, in which devices arranged ex vivo for the guidance and/or movement and/or loading of the carriers are used before the in vivo transfer.

5. The method according to claim 1, in which carriers are used which move autonomously, with multiple carriers moving independently of one another in the same or different directions or moving as a swarm in the same direction.

6. The method according to claim 1, in which the carriers are moved by an external physical field, such as a magnetic or ultrasound field, or by means of chemical, thermal, or physical stimuli.

7. The method according to claim 1 in which the position and movement of the carriers are monitored by imaging methods.

8. The method according to claim 1, in which parameters from the environment of the carriers are detected, transmitted, and monitored in situ by sensors or actuators located on or in the carriers, and/or changes in the parameters are realized based on detected parameters from the environment of the carrier.

9. A device at least for the controlled movement of gametes and/or zygotes and/or embryos in fluids, comprising:
at least one carrier having maximum dimensions in all spatial directions of 500 μm, which carrier is composed at least primarily of biocompatible materials.

10. The device according to claim 9, in which the at least one carrier has dimensions in all spatial directions of 10 nm to 100 μm, advantageously of 1000 nm to 80 μm, more advantageously of 10 nm to 1000 nm.

11. The device according to claim 9, in which the biocompatible materials of the carriers are fully degradable in the body.

12. The device according to claim 9, in which the outer shell of the carriers is entirely composed of biocompatible materials.

13. The device according to claim 9, in which, as biocompatible materials, hydrogels, such as gelatin, methacrylates, collagen, silk, alginates, or biodegradable metal oxides, or metallic alloys such as FeMgSi or FePt, with or without magnetically hard materials such as NdFeB, CrO₂, or BaFe₁₂O₁₉, or superparamagnetic iron oxide nanoparticles, such as SPIONs or iron-platinum nanoparticles are present, or other materials are coated with biocompatible materials, such as surface coatings made of proteins, gold, or polymers such as parylene or poly(ethylene glycol) diacrylate (PEGDA).

14. The device according to claim 9, in which the carriers comprise components that are holders, grippers, slings, catheters, cavities for the releasable connection to gametes and/or zygotes and/or embryos, sensors and/or actuators.

15. The device according to claim 9, in which the carriers comprise propulsion devices for autonomous movement.

16. The device according to claim 9, in which the carriers are microrobots or nanorobots.

17. The device according to claim 9, in which the carriers comprise materials with which the carriers are moved by external or local influences.

18. The device according to claim 17, in which the carriers contain organic, inorganic, and/or magnetic materials.

19. The device according to claim 9, in which, on or in the carriers, sensors or actuators are present which detect, transmit, and monitor in situ parameters from the environment of the carriers and/or realize changes in the parameters based on detected parameters from the environment of the carrier.

20. The device according to claim 9, in which the carrier or carriers is/are connected to devices arranged ex vivo for the guidance and/or movement and/or loading of the carriers before the in vivo transfer, wherein the connection is realized via mechanical devices or digitally.

21. The device according to claim 9, in which the carriers comprise functional materials and/or biomolecules for the protection and/or for the growth of the gametes and/or zygotes and/or embryos.

22. The device according to claim 21, in which the carriers comprise antioxidants, maturation factors, nutrients, protective molecules for reducing thermal and mechanical stresses.
